(19) United States
(12) Patent Application Publication
(10) Pub. No.: US 2025/0387309 A1
(43) Pub. Date: Dec. 25, 2025

(54) METHOD FOR THE TREATMENT OF KERATIN FIBRES COMPRISING A PRETREATMENT OR POST-TREATMENT STEP

(71) Applicant: L'OREAL, PARIS (FR)

(72) Inventors: Nawel BAGHDADLI, AULNAY-SOUS-BOIS (FR); Andrew GREAVES, AULNAY-SOUS-BOIS (FR)

(21) Appl. No.: 18/876,563

(22) PCT Filed: Jun. 21, 2023
(86) PCT No.: PCT/EP2023/066777

§ 371 Date: Dec. 18, 2024

(30) Foreign Application Priority Data
Jun. 22, 2022 (FR) FR2206169

(51) Int. Cl. A61K 8/22; A61K 8/19; A61K 8/25; A61K 8/34; A61K 8/67; A61Q 5/00; A61Q 5/10

(52) U.S. Cl. CPC A61K 8/22; A61K 8/19; A61K 8/25; A61K 8/347; A61K 8/676; A61Q 5/004; A61Q 5/10; A61K 2800/88

(57) ABSTRACT

The present invention relates to a method for the treatment of keratin fibres comprising the application, to the fibres, of a composition comprising at least one chemical oxidizing agent, at least one (bi)carbonate and at least one silicate, this application being preceded and/or followed by the application, to the keratin fibres, of a composition comprising one or more compounds chosen from organic acids, amino acids, chitosans, vitamins B, vitamin C, vanillin, their salts, their solvates and their mixtures.

### TECHNICAL FIELD OF THE INVENTION

The present invention relates to a method for the treatment of keratin fibres comprising the application, to the fibres, of a composition comprising at least one chemical oxidizing agent, at least one (bi)carbonate and at least one silicate, this application being preceded and/or followed by the application, to the keratin fibres, of a composition comprising one or more compounds chosen from organic acids, amino acids, chitosans, vitamins B, vitamin C, vanillin, their salts, their solvates and their mixtures.

### BACKGROUND OF THE INVENTION

When a person wishes to change their hair colour, in particular when they wish to obtain a lighter colour than their original colour, it is often necessary to carry out, beforehand, hair lightening or bleaching. To do this, lightening or bleaching products are used.

The lightening of the hair is evaluated by the “tone depth”, which characterizes the degree or level of lightening. The notion of “tone” is based on the classification of natural shades, one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pp. 215 and 278. The tone depths range from 1 (black) to 10 (very light blond), one unit corresponding to one tone; the higher the figure, the lighter the shade.

It is known practice to lighten or bleach head hair with lightening or bleaching compositions containing at least one chemical oxidizing agent, under alkaline pH conditions in the vast majority of cases. The role of this oxidizing agent is to degrade the melanin of head hair, which, depending on the nature of the oxidizing agent present and on the pH conditions, results in more or less pronounced lightening of the fibres. Thus, for relatively mild lightening, the oxidizing agent is generally hydrogen peroxide. When greater lightening is desired, in particular when the treated head hair is dark, use is normally made of persulfates in the presence of hydrogen peroxide. However, the lightening obtained by the action of such a combination is not always satisfactory because head hair with rather unattractive orangey-yellow shades, which are very different from natural shades, is obtained, which complicates the subsequent colouring by limiting it to the obtaining of warm tones. Furthermore, persulfate-based lightening compositions can lead to a degradation of the quality and integrity of the fibre. The hair can then end up deformed or damaged, which in particular results in significant breakage during combing or disentangling.

Thus, in order to overcome these disadvantages, it is common practice to employ hair care compositions intended for conditioning the hair by giving it satisfactory cosmetic properties, in particular smoothness, sheen, softness to the touch, suppleness and lightness, and also good disentangling properties leading to easy combing and good manageability of the hair, which is thus easier to style and holds its shaping. However, the conditioning effect obtained by these hair care compositions fades out rapidly over time and does not make it possible to strengthen the hair cortex, in particular by improving the density of bondings between the proteins present in the cortex of the individual hair, in order to protect or repair the individual hair and to reduce the breakage thereof, for example during combing or disentangling. Furthermore, these care compositions can, in certain cases, detrimentally affect the dyeing or bleaching of the hair thus treated.

There thus exists a real need to develop a method which makes it possible to obtain efficient lightening of keratin fibres, in particular of dark keratin fibres, with a less yellow and more natural result. Such a method should also be more respectful of the quality and the integrity of the fibres and make it possible in particular to retain, indeed even improve, the quality and integrity of the keratin fibres and in particular to reduce the breakage thereof, while avoiding detrimentally affecting the lightening, in particular the deyellowing, of the fibres thus treated.,

The Applicant Company has discovered, surprisingly, that all or some of these objectives can be achieved by the method according to the present invention.

### SUMMARY OF THE INVENTION

According to a first aspect, a subject-matter of the present invention is a method for the treatment of keratin fibres comprising the following step b) and also the following step a) and/or the following step c):
a) application to the keratin fibres of a composition (A) comprising one or more compounds D chosen from organic acids, amino acids, chitosans, vitamins B, vitamin C, vanillin, their salts, their solvates and their mixtures, preferably chosen from organic acids, amino acids, chitosans, vitamins B, their salts, their solvates and their mixtures, the compound(s) D being present in the composition (A) in a total content by weight ranging from 1% to 20% by weight, with respect to the total weight of the composition (A);
b) application to the keratin fibres of a composition (B) comprising:
i) one or more chemical oxidizing agents chosen from hydrogen peroxide, hydrogen peroxide-generating systems other than peroxygenated salts, and their mixtures;
ii) one or more compounds chosen from carbonates, carbonate-generating systems, bicarbonates, bicarbonate-generating systems and their mixtures, preferably from carbonates, bicarbonates and their mixtures;
iii) one or more silicates;
c) application to the keratin fibres of a composition (C) comprising one or more compounds D as defined above, the compound(s) D being present in the composition (C) in a total content by weight ranging from 1% to 25% by weight, with respect to the total weight of the composition (C);
in which:
   when step a) is present, step a) is carried out before step b);
   when step c) is present, step c) is carried out after step b);
   the compositions (A) and (C) can be identical or different;
   the composition (B) comprises a total content of persulfates of less than 10% by weight.

According to a second aspect, a subject-matter of the present invention is a composition (B) as defined above comprising one or more compounds chosen from chitosans, vitamins B, vitamin C, vanillin, their salts, their solvates and their mixtures, preferably chosen from chitosans, vitamins B, their salts, their solvates and their mixtures.

According to a third aspect, a subject-matter of the present invention is the use of the composition(s) (A) and/or (C) as defined above for reducing the damage to keratin fibres treated by a method for the treatment of keratin fibres employing the composition (B) as defined above.

According to a fourth aspect, a subject-matter of the present invention is a multicompartment device (kit) comprising:
a first compartment including a composition (B1) comprising:
one or more chemical oxidizing agents chosen from hydrogen peroxide, hydrogen peroxide-generating systems other than peroxygenated salts and their mixtures as defined above, preferably hydrogen peroxide; and
a second compartment including a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
optionally one or more colouring agents, preferably chosen from direct dyes, oxidation dyes and their mixtures; and
optionally one or more compounds D as defined above; and
a third compartment including a composition (A) or (C) as defined above, it being possible for the composition (A) or (C) to optionally comprise one or more colouring agents, preferably chosen from direct dyes, oxidation dyes and their mixtures; and
optionally a fourth compartment including a composition (B3) comprising one or more colouring agents, preferably chosen from direct dyes, oxidation dyes and their mixtures.

### DETAILED DESCRIPTION OF THE INVENTION

Within the meaning of the present invention and unless otherwise indicated:
The term “keratin fibres” is understood to mean fibres of human or animal origin, such as head hair, body hairs, the eyelashes, the eyebrows, wool, angora, cashmere or fur.

According to the present invention, the keratin fibres are preferably human keratin fibres, more preferentially the hair.
The term “salt” is understood to mean an addition salt with an organic or inorganic acid or base. The addition salts with an acid are in particular chosen from the addition salts with acids such as hydrochloric acid, hydrobromic acid, sulfuric acid, citric acid, succinic acid, tartaric acid, lactic acid, tosylic acid, benzenesulfonic acid, phosphoric acid or acetic acid. The addition salts with a base are in particular chosen from the addition salts with bases such as the basifying agents defined below, in particular alkali metal hydroxides, alkaline earth metal hydroxides, aqueous ammonia, amines or alkanolamines.
The term “alkyl group” is understood to mean a saturated, linear or branched, hydrocarbon radical comprising from 1 to 30 carbon atoms, preferentially from 1 to 26 carbon atoms, more preferentially from 1 to 22 carbon atoms, for example methyl, ethyl, n-propyl, isopropyl, butyl, n-pentyl, n-hexyl, n-decyl, n-dodecyl, tetradecyl, hexadecyl or eicosyl.
The term “(Cₓ-C_{y})alkyl group” is understood to mean an alkyl group comprising from x to y carbon atoms.
The term “alkenyl group” is understood to mean a linear or branched hydrocarbon radical comprising one or more conjugated or non-conjugated ethylenic unsaturations and from 1 to 30 carbon atoms, preferably from 1 to 26 carbon atoms, more preferentially from 1 to 22 carbon atoms, for example ethylenyl, n-propylenyl, isopropylenyl, butylenyl, n-pentylenyl, n-hexylenyl, n-decylenyl, n-dodecylenyl, tetradecylenyl, hexadecylenyl or eicosylenyl.
The term “(Cₓ-C_{y})alkenyl group” is understood to mean an alkenyl group comprising from x to y carbon atoms.
The term “alkoxy group” is understood to mean an alkyl group bonded to an oxygen atom.
The term “(Cₓ-C_{y})alkoxy group” is understood to mean an alkoxy group comprising from x to y carbon atoms.
The term “(Cₓ-C_{y})hydroxyalkyl group” is understood to mean a (Cₓ-C_{y})alkyl group, one at least of the hydrogen atoms of which is replaced by a hydroxyl (—OH) group.
The term “alkylamino group” is understood to mean an R′—NR^{a}— group with R′ representing an alkyl group and R^{a} representing a hydrogen atom or a (C₁-C₄)alkyl group optionally substituted by one or more —X(O)OH groups with X representing a carbon atom or S═O or P—OH, preferably with X representing a carbon atom; it being possible for R′ and R^{a} to form, with the nitrogen atom which carries them, a saturated or unsaturated, 4- to 8-membered, preferably 5- or 6-membered, cyclic group, said cyclic group optionally being substituted, in particular by one or more identical or different groups chosen from (C₁-C₄)(poly)hydroxyalkyl or (C₁-C₄)alkoxy groups.
The term “alkylamido group” is understood to mean an R″—NR^{a}—CO— group with R″ representing an alkyl group and R^{a} representing a hydrogen atom or a (C₁-C₄)alkyl group optionally substituted by one or more —X(O)OH groups with X representing a carbon atom or S═O or P—OH, preferably with X representing a carbon atom; it being possible for R″ and R^{a} to form, with the nitrogen atom which carries them, a saturated or unsaturated, 4- to 8-membered, preferably 5- or 6-membered, cyclic group, said cyclic group optionally being substituted, in particular by one or more identical or different groups chosen from (C₁-C₄)(poly)hydroxyalkyl or (C₁-C₄)alkoxy groups.
The term “(hetero)cyclic group” is understood to mean a cyclic or heterocyclic group.
The term “cyclic group” is understood to mean a saturated or unsaturated, in particular aromatic, fused or non-fused, mono- or polycyclic carbocycle comprising from 6 to 22 carbon atoms, it being possible for said cyclic group to be substituted by one or more identical or different groups in particular chosen from: (C₁-C₆)alkyl, (C₁-C₆)alkoxy, (C₁-C₆)hydroxyalkyl, hydroxyl (—OH) or carboxyl (—COOH).
The term “heterocyclic group” is understood to mean a saturated or unsaturated, in particular aromatic, fused or non-fused, mono- or polycyclic group comprising from 5 to 22 ring members and including from 1 to 3 heteroatoms chosen from the nitrogen, oxygen or sulfur atom, it being possible for said heterocyclic group to be substituted by one or more identical or different groups in particular chosen from: (C₁-C₆)alkyl, (C₁-C₆)alkoxy, (C₁-C₆)hydroxyalkyl, hydroxyl (—OH) or carboxyl (—COOH).
The term “amino acid” is understood to mean an organic compound comprising at least one carboxyl (—COOH) or carboxylate (—COO⁻) or sulfo (—SO₂OH) or sulfonate (—SO₂O⁻) or sulfinyl (—SO₂H) or sulfinate (—SO₂⁻) group and at least one primary, secondary or tertiary amino group or one quaternary ammonium group.
The term “silicate” is understood to mean a salt of a silicic acid.
The term “colouring agent” is understood to mean an oxidation dye, a direct dye or a pigment.
The term “oxidation dye” is understood to mean an oxidation dye precursor chosen from oxidation bases and couplers. Oxidation bases and couplers are colourless or sparingly coloured compounds which, by a condensation reaction in the presence of an oxidizing agent, give a coloured entity.
The term “direct dye” is understood to mean any natural and/or synthetic dye, including in the form of extract(s), other than oxidation dyes. These are coloured compounds that will diffuse superficially over the fibre. They can be ionic or non-ionic, i.e. anionic, cationic, neutral or non-ionic.
The term “chemical oxidizing agent” is understood to mean an oxidizing agent other than atmospheric oxygen.

Unless otherwise indicated, when compounds are mentioned in the present patent application, this is also understood to mean their optical isomers, their geometrical isomers, their tautomers, their salts, their solvates, such as hydrates, and their mixtures. The expressions “at least one” and “one or more” are synonymous and can be used interchangeably.

The expressions “lightening” and “bleaching” are synonymous and can be used interchangeably.

### Method

According to a first aspect, a subject-matter of the present invention is a method for the treatment of keratin fibres comprising step b) and also step a) and/or step c) as defined above.

Steps a), b) and c) are separate.

According to a preferred embodiment, the method according to the present invention is a method for lightening keratin fibres.

The Applicant Company has found, surprisingly, that the method according to the present invention makes it possible to obtain efficient lightening of keratin fibres with a less yellow and more natural result.

When the colour of the keratin fibres treated by the method according to the invention is compared with the colour of the keratin fibres treated by methods employing lightening compositions known from the state of the art, values of b*, measured in the CIE L*a*b* system, are observed which are lower for the method according to the invention than for the methods employing lightening compositions known from the state of the art, at equivalent intensity level L*.

Furthermore, the method according to the present invention is more respectful of the quality of the fibres and makes it possible in particular to retain, indeed even improve, the quality of the keratin fibres and in particular to reduce the breakage thereof, while avoiding detrimentally affecting the lightening, in particular the deyellowing, of the fibres thus treated.

In the method according to the present invention, it is essential for steps a) and b) to be successive, that is to say that, when step a) is present, for step a) to be carried out before step b).

In the method according to the present invention, it is essential for steps b) and c) to be successive, that is to say that, when step c) is present, for step b) to be carried out before step c).

The method can, however, comprise one or more additional steps between steps a) and b) and/or between steps b) and c).

### Steps a) and c)

The method according to the invention comprises step a) and/or step c) as are defined above.

According to a preferred embodiment, the method comprises step a) as defined above.

According to another preferred embodiment, the method comprises step c) as defined above.

According to another preferred embodiment, the method comprises step a) and step c) as are defined above.

### Compositions (A) and (C)

The compositions (A) and (C) comprise one or more compounds D chosen from organic acids, amino acids, chitosans, vitamins B, vitamin C, vanillin, their salts, their solvates and their mixtures.

The compositions (A) and (C) can be identical or different.

### Compounds D

The compound(s) D are present in the composition (A) in a total content by weight preferably ranging from 3% to 20% by weight, with respect to the total weight of the composition (A).

The compound(s) D are present in the composition (C) in a total content by weight preferably ranging from 2% to 15% by weight, with respect to the total weight of the composition (C).

According to a preferred embodiment, the compound(s) D are chosen from organic acids, their salts, their solvates and their mixtures.

According to another preferred embodiment, the compound(s) D are chosen from amino acids, their salts, their solvates and their mixtures.

According to another preferred embodiment, the compound(s) D are chosen from chitosans, their salts, their solvates and their mixtures.

According to another preferred embodiment, the compound(s) D are chosen from vitamins B, their salts, their solvates and their mixtures.

According to another preferred embodiment, the compound(s) D are chosen from vitamin C, its salts, its solvates and their mixtures.

According to a particularly preferred embodiment, the compound D is chosen from vanillin, its solvates and their mixtures.

According to a particularly preferred embodiment, the compound(s) D are chosen from organic acids, amino acids, their salts, their solvates and their mixtures.

According to a more particularly preferred embodiment, the compound(s) D are chosen from mixtures of organic acids and amino acids, their salts, their solvates and their mixtures.

By way of example, the compounds D are chosen from mixtures of glycine and citric acid.

### Organic Acids

Preferably, the organic acids are chosen from the compounds of following formula (I):

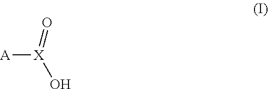

in which formula (I):
X represents a carbon atom or S═O or P—OH, preferably a carbon atom;
A represents an —OR⁴ group or a —CR¹(R²)ₚ(R³) group, it being understood that, if X represents P—OH, then A represents an —OR⁴ group;
p=0 or 1;
R¹ represents a hydrogen atom or a hydroxyl (—OH) group or an —NR^{a}—R^{c} group or an —NR^{a}—CO—R^{c} group;
R² represents a hydrogen atom or a hydroxyl (—OH) group or an —X(O)OH group with X as defined above, in particular with X representing a carbon atom;
R³ represents a hydrogen atom or a hydroxyl (—OH) group or a (hetero)cyclic group optionally substituted by one or more identical or different groups chosen from (C₁-C₆)alkyl, in particular (C₁-C₄)alkyl, such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, in particular (C₁-C₄)alkoxy, such as methoxy, —X(O)OH with X as defined above, in particular with X representing a carbon atom, or —O—X(O)OH with X as defined above or R³ represents an alkyl group or an alkenyl group or an alkylamino group or an alkylamido group, said alkyl or alkenyl or alkylamino or alkylamido group being:
optionally substituted by one or more identical or different groups chosen from an —X(O)OH group with X as defined above, in particular with X representing a carbon atom;
a hydroxyl (—OH) group or a (hetero)cyclic group; and/or
optionally interrupted by one or more heteroatoms or groups chosen from —O—, —NR^{a}—, —S—, —CO—, or their combinations, such as —O—CO—, —(CO)—O—, —NR^{a}—(CO)— or —(CO)—NR^{a}—;
   it being possible for R¹ and R³ to form, together with the carbon atom which carries them, a (hetero)cyclic group optionally substituted by one or more identical or different groups chosen from: (C₁-C₆)alkyl, in particular (C₁-C₄)alkyl, such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, in particular (C₁-C₄)alkoxy, such as methoxy, —X(O)OH with X as defined above, in particular with X representing a carbon atom, or —O—X(O)OH with X as defined above, in particular with X=P—OH;
   it being possible for R¹ and R³ to form, together with the carbon atom which carries them, a carbonyl —CO— group;
R⁴ represents a hydrogen atom or an alkyl group or an alkenyl group or a (hetero)cyclic group, said alkyl or alkenyl or (hetero)cyclic group being optionally substituted by one or more identical or different groups chosen from: an —X(O)OH group with X as defined above, in particular with X representing a carbon atom or P—OH; a hydroxyl (—OH) group or a (hetero)cyclic group;
R^{a} represents a hydrogen atom or a (C₁-C₄)alkyl group optionally substituted by one or more —X(O)OH groups with X as defined above, in particular with X representing a carbon atom;
R^{c} represents an alkyl group or an alkenyl group or an alkylamino group or an alkylamido group, said alkyl or alkenyl or alkylamino or alkylamido group being:
optionally substituted by one or more identical or different groups chosen from: an —X(O)OH group with X as defined above, in particular with X representing a carbon atom;
   a hydroxyl (—OH) group or a (hetero)cyclic group; and/or
optionally interrupted by one or more heteroatoms or groups chosen from —O—, —S—, —CO—, —O—CO—, —O—CO—, —NR^{a}—(CO)— or —(CO)—NR^{a}—.

According to a specific embodiment of the invention, the organic acids are chosen from the compounds of formula (I) in which X represents a carbon atom, A represents a —CR¹(R²)ₚ(R³) group in which p=1, R¹ represents a hydrogen atom or an —NR^{a}—CO—R^{c} group with R^{a} and R^{c} as defined above, preferably R^{a} denotes a hydrogen atom and R^{c} denotes an alkyl radical, R² represents a hydrogen atom or a hydroxyl (—OH) group, R³ represents a (hetero)cyclic group optionally substituted by one or more identical or different groups chosen from (C₁-C₆)alkyl, in particular (C₁-C₄)alkyl, such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, in particular (C₁-C₄)alkoxy, such as methoxy, or —C(O)OH or R³ represents an alkyl group or an alkenyl group, said alkyl or alkenyl group being:
optionally substituted by one or more identical or different groups chosen from a —C(O)OH group; a hydroxyl (—OH) group; and/or
optionally interrupted by —CO—, —O—CO—, —(CO)—O—, —NR^{a}—(CO)—, —(CO)—NR^{a}— with R^{a} as defined above, preferably optionally interrupted by —CO—, —O—CO—, —(CO)—O—.

According to a specific embodiment of the invention, the organic acids are chosen from the compounds of formula (I) in which X represents a carbon atom, A represents a —CR¹(R²)(R³) group in which R¹ represents a hydrogen atom, R² represents a hydrogen atom or a hydroxyl (—OH) group, R³ represents a (hetero)cyclic group, preferably an aromatic hydrocarbon group, such as phenyl, said (hetero)cyclic group being optionally substituted by one or more identical or different groups chosen from: (C₁-C₆)alkyl, in particular (C₁-C₄)alkyl, such as methyl, (C₁-C₆)alkoxy, in particular (C₁-C₄)alkoxy, such as methoxy, or —C(O)OH or R³ represents an alkyl group or an alkenyl group optionally interrupted by —NR^{a}—(CO)— or —(CO)—NR^{a}— with R^{a} as defined above, preferably not interrupted, and/or optionally substituted by a hydroxyl group.

According to another specific embodiment of the invention, the organic acids are chosen from the compounds of formula (I) in which X represents a carbon atom, A represents a —CR¹(R²)(R³) group in which R¹ represents a hydrogen atom or an —NR^{a}—CO—R^{c} group with R^{a} and R^{c} as defined above, preferably R^{a} represents a hydrogen atom and R^{c} represents an alkyl radical, R² represents a hydrogen atom or a hydroxyl (—OH) group, R³ represents an alkyl group or an alkenyl group, said alkyl or alkenyl group being substituted by one or more —C(O)OH groups, in particular substituted by one or two —C(O)OH groups, and:
optionally substituted by one or more hydroxyl (—OH) groups; and/or
optionally interrupted by —CO—, —O—CO—, —(CO)—O—, —(CO)—NR^{a}—.

According to another specific embodiment of the invention, the organic acids are chosen from the compounds of formula (I) in which X represents a carbon atom, A represents a —CR¹(R³) group in which R¹ and R³ form, together with the carbon atom which carries them, a (hetero)cyclic group, which is preferably aromatic, optionally substituted by one or more identical or different groups chosen from: (C₁-C₆)alkyl, in particular (C₁-C₄)alkyl, such as methyl, alkylcarbonyl —(CO)—R^{c}, alkylcarbonyloxy —O—CO—R^{c}, alkyloxycarbonyl —CO—O—R^{c}, —OH, (C₁-C₆)alkoxy, in particular (C₁-C₄)alkoxy, such as methoxy, —C(O)OH, with R^{c} as defined above.

According to a preferred embodiment, the organic acids are chosen from the compounds of formula (I) in which:
X represents a carbon atom;
A represents a —CR¹(R²)ₚ(R³) group;
p=1;
R¹ represents a hydrogen atom or a hydroxyl (—OH) group;
R² represents a hydrogen atom or a hydroxyl (—OH) group or a —C(O)OH group;
R³ represents a hydrogen atom or a hydroxyl (—OH) group or an alkyl group or an alkenyl group or an alkylamino group or an alkylamido group, said alkyl or alkenyl or alkylamino or alkylamido group being:
optionally substituted by one or more identical or different groups chosen from a —C(O)OH group; a hydroxyl (—OH) group or a (hetero)cyclic group; and/or
optionally interrupted by one or more heteroatoms or groups chosen from —O—, —NR^{a}—, —S—, —CO—, or their combinations, such as —O—CO—, —(CO)—O—, —NR^{a}—(CO)— or —(CO)—NR^{a}—;
R^{a} represents a hydrogen atom or a (C₁-C₄)alkyl group optionally substituted by one or more —C(O)OH groups.

According to a more preferred embodiment, the organic acids are chosen from levulinic acid, N,N-bis(carboxymethyl)glutamic acid, oleic acid, citric acid, lactic acid, maleic acid, succinic acid, glutaric acid, tartaric acid, adipic acid and their mixtures.

The N,N-bis(carboxymethyl)glutamic acid is preferably N,N-bis(carboxymethyl)-L-glutamic acid.

According to an even more preferred embodiment, the organic acids are chosen from levulinic acid, N,N-bis(carboxymethyl)glutamic acid, oleic acid, citric acid and their mixtures.

According to a most preferred embodiment, the organic acids are chosen from levulinic acid, N,N-bis(carboxymethyl)-L-glutamic acid, tetrasodium N,N-bis(carboxymethyl)-L-glutamate, oleic acid, citric acid and their mixtures.

### Amino Acids

The amino acids can be chosen from acidic amino acids, basic amino acids, polar neutral amino acids, non-polar neutral amino acids and their mixtures.

The term “acidic amino acid” is understood to mean an amino acid comprising more acidic functions than basic functions, in particular proteinogenic amino acids, the side chain of which comprises an acidic function. Mention may be made, by way of example of acidic amino acid, of aspartic acid, glutamic acid and their mixtures.

The term “basic amino acid” is understood to mean an amino acid comprising more basic functions than acidic functions, in particular proteinogenic amino acids, the side chain of which comprises a basic function. Mention may be made, by way of example of basic amino acid, of arginine, histidine, lysine and their mixtures.

The term “polar neutral amino acid” is understood to mean an amino acid which is uncharged at pH=7 and which comprises a polar chemical function, such as a thiol, alcohol or phenol, or basic amide CONH₂ function. Mention may be made, by way of example of polar neutral amino acid, of serine, threonine, cysteine, asparagine, glutamine, tyrosine and their mixtures.

The term “non-polar neutral amino acid” is understood to mean an amino acid which is uncharged at pH=7 and non-polar. Mention may be made, by way of example of non-polar neutral amino acid, of an amino acid chosen from glycine, alanine, valine, leucine, isoleucine, methionine, phenylalanine, tryptophan, proline and their mixtures.

The amino acids are preferably chosen from glycine, proline, methionine, serine, arginine, lysine, carnitine, acetylcarnitine, propionylcarnitine, isovalerylcarnitine, taurine, hypotaurine, creatinine, carnosine, aspartame and their mixtures.

The amino acids are more preferentially chosen from glycine, proline, methionine, serine, arginine, lysine, carnitine and their mixtures.

The amino acids are more preferentially still chosen from glycine, arginine, carnitine and their mixtures.

According to a preferred embodiment, the amino acids are chosen from glycine, L-arginine and their mixtures.

### Vitamins B

Preferably, the vitamins B are chosen from vitamin B1, vitamin B3, vitamin B6 and their mixtures.

The term “vitamin 1” is understood to mean thiamine.

The term “vitamin B3” is understood to mean nicotinic acid or nicotinamide.

The term “vitamin B6” is understood to mean pyridoxine, pyridoxal and pyridoxamine.

More preferentially, the vitamins B are chosen from vitamin B1, vitamin B6 and their mixtures.

More preferentially still, the vitamins B are chosen from thiamine, pyridoxine, their salts and their mixtures.

Preferably, the vitamins B are chosen from thiamine salts, pyridoxine salts and their mixtures.

Mention may be made, byway of example of thiamine salt which can be used, of thiamine hydrochloride.

Mention may be made, by way of example of pyridoxine salt which can be used, of pyridoxine hydrochloride.

According to a preferred embodiment, the composition (A) comprises a total content of colouring agents of less than 0.1% by weight, preferably of less than 0.01% by weight, more preferentially of less than 0.001% by weight, with respect to the total weight of the composition (A), and/or the composition (C) comprises a total content of colouring agents of less than 0.1% by weight, preferably of less than 0.01% by weight, more preferentially of less than 0.001% by weight, with respect to the total weight of the composition (C). According to a more preferred embodiment, the composition (A) and/or (C) is devoid of colouring agents.

### pH of the Compositions (A) and (C)

The pH of the composition (A) is preferably greater than 7, more preferentially ranging from 7 to 10, more preferentially still ranging from 7.5 to 9.5.

The pH of the composition (C) is preferably less than 7, more preferentially ranging from 3 to 7, more preferentially still ranging from 3.1 to 6.5, most preferentially ranging from 3.5 to 6.

### Composition (B)

According to a preferred embodiment, the composition (B) comprises:
i) hydrogen peroxide;
ii) one or more compounds chosen from carbonates, bicarbonates and their mixtures;
iii) one or more silicates.

### Chemical Oxidizing Agents

The composition (B) comprises i) one or more chemical oxidizing agents chosen from hydrogen peroxide, hydrogen peroxide-generating systems other than peroxygenated salts, and their mixtures.

The hydrogen peroxide-generating systems other than peroxygenated salts can be chosen from urea peroxide, polymeric complexes which can release hydrogen peroxide, oxidases and their mixtures.

Mention may be made, by way of example of polymeric complexes which can release hydrogen peroxide, of polyvinylpyrrolidone/H₂O₂, in particular which is provided in the powder form, and the other polymeric complexes described in U.S. Pat. Nos. 5,008,093, 3,376,110 and 5,183,901.

Oxidases can produce hydrogen peroxide in the presence of a suitable substrate, such as, for example, glucose in the case of glucose oxidase or uric acid with uricase.

According to a specific embodiment, hydrogen peroxide and/or the hydrogen peroxide-generating system(s) other than peroxygenated salts can be incorporated in the composition according to the invention just before its application to the keratin fibres. The intermediate composition(s) comprising the hydrogen peroxide and/or the hydrogen peroxide-generating system(s) other than peroxygenated salts can be referred to as oxidizing compositions and can also include various additional compounds or various adjuvants conventionally used in compositions for lightening keratin fibres.

According to a preferred embodiment, the composition (B) comprises hydrogen peroxide as chemical oxidizing agent.

The chemical oxidizing agent(s) are present in the composition (B) preferably in a total content ranging from 1% to 12% by weight, more preferentially ranging from 3% to 9% by weight, more preferentially still ranging from 3.5% to 8.5% by weight, with respect to the total weight of the composition (B).

According to a preferred embodiment, the hydrogen peroxide is present in the composition (B) in a total content ranging from 1% to 12% by weight, preferably ranging from 3% to 9% by weight, more preferentially ranging from 3.5% to 8.5% by weight, with respect to the total weight of the composition (B).

(Bi)Carbonates and/or (Bi)Carbonate-Generating Systems

The composition (B) additionally comprises ii) one or more compounds chosen from carbonates, carbonate-generating systems, bicarbonates, bicarbonate-generating systems and their mixtures.

According to a preferred embodiment, the composition (B) additionally comprises ii) one or more compounds chosen from carbonates, bicarbonates and their mixtures.

According to a more preferred embodiment, the composition (B) additionally comprises ii) one or more compounds chosen from ammonium carbonate, ammonium bicarbonate and their mixtures.

The compound(s) ii) are present in the composition (B) preferably in a total content ranging from 0.01% to 20% by weight, preferably ranging from 1% to 15% by weight, more preferentially ranging from 2% to 15% by weight, more preferentially still ranging from 4% to 15% by weight, with respect to the total weight of the composition (B).

Carbonates and/or Carbonate-Generating Systems

The term “carbonate-generating system” is understood to mean a system which generates the carbonate in situ, such as, for example, carbon dioxide in water or percarbonate in water.

Preferably, the carbonate(s) are chosen from:
alkali metal carbonates;
alkaline earth metal carbonates;
lanthanide carbonates;
transition metal carbonates;
bismuth carbonate;
cadmium carbonate;
thallium carbonate;
zinc carbonate;
the compounds of formula (N⁺R¹R²R³R⁴)₂CO₃²⁻ in which R¹, R², R³ and R⁴ represent, independently of one another, a hydrogen atom or a (C₁-C₄)alkyl group optionally substituted by a hydroxyl group;
guanidine carbonate;
their mixtures.

More preferentially, the carbonate(s) are chosen from sodium carbonate, potassium carbonate, caesium carbonate, lithium carbonate, magnesium carbonate, calcium carbonate, barium carbonate, strontium carbonate, cerium carbonate, lanthanum carbonate, yttrium carbonate, copper(II) carbonate, manganese carbonate, nickel carbonate, silver carbonate, zirconium carbonate, bismuth carbonate, cadmium carbonate, thallium carbonate, zinc carbonate, ammonium carbonate, guanidine carbonate, tetraethylammonium carbonate and their mixtures.

More preferentially still, the carbonate(s) are chosen from sodium carbonate, potassium carbonate, caesium carbonate, magnesium carbonate, calcium carbonate, cerium carbonate, manganese carbonate, zinc carbonate, ammonium carbonate, guanidine carbonate and their mixtures.

Most preferentially, the carbonate(s) are chosen from sodium carbonate, potassium carbonate, magnesium carbonate, calcium carbonate, ammonium carbonate and their mixtures.

According to a particularly preferred embodiment, the carbonate included in the composition (B) is ammonium carbonate.

The carbonate(s) and/or the carbonate-generating system(s) are present in the composition (B) preferably in a total content ranging from 0.01% to 20% by weight, more preferentially ranging from 1% to 20% by weight, more preferentially still ranging from 1% to 10% by weight, with respect to the total weight of the composition (B).

According to a preferred embodiment, the carbonate(s) are present in the composition (B) in a total content ranging from 0.01% to 20% by weight, preferably ranging from 1% to 20% by weight, more preferentially ranging from 1% to 10% by weight, with respect to the total weight of the composition (B).

According to a preferred embodiment, the compound(s) ii) are chosen from carbonates, carbonate-generating systems and their mixtures, preferably from carbonates.

Bicarbonates and/or Bicarbonate-Generating Systems

The term “bicarbonate-generating system” is understood to mean a system which generates the bicarbonate in situ, such as, for example, carbon dioxide in water or by buffering a carbonate with an inorganic or organic acid.

Preferably, the bicarbonate(s) are chosen from:
alkali metal bicarbonates;
alkaline earth metal bicarbonates;
the compounds of formula N⁺R¹R²R³R⁴HCO₃ in which R¹, R², R³ and R⁴ represent, independently of one another, a hydrogen atom or a (C₁-C₄)alkyl group optionally substituted by a hydroxyl group;
aminoguanidine bicarbonate;
their mixtures.

More preferentially, the bicarbonate(s) are chosen from sodium bicarbonate, potassium bicarbonate, lithium bicarbonate, caesium bicarbonate, calcium bicarbonate, magnesium bicarbonate, ammonium bicarbonate, choline bicarbonate, triethylammonium bicarbonate, aminoguanidine bicarbonate and their mixtures.

More preferentially still, the bicarbonate(s) are chosen from sodium bicarbonate, potassium bicarbonate, caesium bicarbonate, calcium bicarbonate, magnesium bicarbonate, ammonium bicarbonate and their mixtures.

Most preferentially, the bicarbonate(s) are chosen from sodium bicarbonate, potassium bicarbonate, ammonium bicarbonate and their mixtures.

According to a particularly preferred embodiment, the bicarbonate included in the composition (B) is ammonium bicarbonate.

The bicarbonates can originate from a natural water, for example spring water from the Vichy basin or from La Roche Posay or Badoit water.

The bicarbonate(s) and/or the bicarbonate-generating system(s) are present in the composition (B) preferably in a total content ranging from 0.01% to 20% by weight, more preferentially ranging from 1% to 15% by weight, more preferentially still ranging from 2% to 15% by weight, most preferentially ranging from 4% to 15% by weight, with respect to the total weight of the composition (B).

According to a preferred embodiment, the bicarbonate(s) are present in a total content ranging from 0.01% to 20% by weight, preferably ranging from 1% to 15% by weight, more preferentially ranging from 2% to 15% by weight, more preferentially still ranging from 4% to 15% by weight, with respect to the total weight of the composition (B).

According to a preferred embodiment, the compound(s) ii) are chosen from bicarbonates, bicarbonate-generating systems and their mixtures, preferably from bicarbonates.

### Silicates

The composition (B) additionally comprises iii) one or more silicates.

The silicate(s) are preferably water-soluble.

The term “water-soluble silicate” is understood to mean a silicate which exhibits a solubility in water, at ordinary ambient temperature (25° C.) and at atmospheric pressure (760 mmHg), of greater than 0.5% by weight, preferably of greater than 1% by weight.

Preferably, the silicate(s) are chosen from alkali metal silicates, alkaline earth metal silicates, aluminium silicates, trimethylammonium silicates and their mixtures. More preferentially, the silicate(s) are chosen from sodium silicates, potassium silicates, calcium silicates, aluminium silicates, trimethylammonium silicates and their mixtures. More preferentially still, the silicate(s) are chosen from sodium silicates.

Preferably, the silicate(s) are chosen from the compounds having the INCI name(s) Sodium Silicate (CAS: [1344-09-8]) and/or Sodium Metasilicate (CAS: [6834-92-0]).

The silicate(s) are present in the composition (B) preferably in a total content ranging from 1% to 40% by weight, more preferentially ranging from 2% to 35% by weight, more preferentially still ranging from 3% to 35% by weight, most preferentially ranging from 4% to 20% by weight, with respect to the total weight of the composition (B).

The total amount of carbonate(s) and/or carbonate-generating system(s)/total amount of silicate(s) ratio by weight in the composition (B) is preferably from 0.00025 to 20, more preferentially from 0.028 to 10, more preferentially still from 0.028 to 3.4.

According to a preferred embodiment, the total amount of carbonate(s)/total amount of silicate(s) ratio by weight in the composition (B) is from 0.00025 to 20, preferably from 0.028 to 10, more preferentially from 0.028 to 3.4.

The total amount of carbonate(s) and/or carbonate-generating system(s)/total amount of chemical oxidizing agent(s) ratio by weight in the composition (B) is preferably from 0.0008 to 20, more preferentially from 0.1 to 6.6, more preferentially still from 0.1 to 2.9.

According to a preferred embodiment, the total amount of carbonate(s)/total amount of chemical oxidizing agent(s) ratio by weight in the composition (B) is from 0.0008 to 20, preferably from 0.1 to 6.6, more preferentially from 0.1 to 2.9.

According to a preferred embodiment, the total amount of carbonate(s)/total amount of hydrogen peroxide ratio by weight in the composition (B) is from 0.0008 to 20, preferably from 0.1 to 6.6, more preferentially from 0.1 to 2.9.

The total amount of bicarbonate(s) and/or bicarbonate-generating system(s)/total amount of silicate(s) ratio by weight in the composition (B) is preferably from 0.00025 to 20, more preferentially from 0.02 to 7.5, more preferentially still from 0.05 to 5.

According to a preferred embodiment, the total amount of bicarbonate(s)/total amount of silicate(s) ratio by weight in the composition (B) is from 0.00025 to 20, preferably from 0.02 to 7.5, more preferentially from 0.05 to 5.

The total amount of bicarbonate(s) and/or bicarbonate-generating system(s)/total amount of chemical oxidizing agent(s) ratio by weight in the composition (B) is preferably from 0.0008 to 20, more preferentially from 0.11 to 5, more preferentially still from 0.2 to 4.2.

According to a preferred embodiment, the total amount of bicarbonate(s)/total amount of chemical oxidizing agent(s) ratio by weight in the composition (B) is from 0.0008 to 20, preferably from 0.11 to 5, more preferentially from 0.2 to 4.2.

According to a more preferred embodiment, the total amount of bicarbonate(s)/total amount of hydrogen peroxide ratio by weight in the composition (B) is from 0.0008 to 20, preferably from 0.11 to 5, more preferentially from 0.2 to 4.2.

The total amount of carbonate(s) and/or carbonate-generating system(s)/total amount of bicarbonate(s) and/or bicarbonate-generating system(s) ratio by weight in the composition (B) is preferably from 0.0005 to 2000, more preferentially from 0.06 to 20, more preferentially still from 0.06 to 5.

According to a preferred embodiment, the total amount of carbonate(s)/total amount of bicarbonate(s) ratio by weight in the composition (B) is from 0.0005 to 2000, preferably from 0.06 to 20, more preferentially from 0.06 to 5.

The composition (B) preferably comprises a total content of magnesium carbonate of less than 5% by weight, more preferentially of less than 1% by weight, more preferentially still of less than 0.1% by weight, most preferentially of less than 0.01% by weight and better still of less than 0.001% by weight.

According to a particularly preferred embodiment, the composition is devoid of magnesium carbonate.

The composition (B) comprises a total content of persulfates of less than 10% by weight, preferably of less than 5% by weight, more preferentially of less than 1% by weight, more preferentially still of less than 0.1% by weight, most preferentially of less than 0.01% by weight and better still of less than 0.001% by weight.

According to a particularly preferred embodiment, the composition is devoid of persulfates.

According to a specific embodiment, the composition (B) additionally comprises iv) one or more colouring agents, preferably chosen from direct dyes, oxidation dyes and their mixtures.

According to this specific embodiment, the method according to the invention is a method for simultaneous bleaching and dyeing of keratin fibres.

When they are present in the composition (B), the colouring agent(s) are present in the composition (B) preferably in a total content ranging from 0.001% to 10% by weight, more preferentially ranging from 0.01% to 4% by weight, more preferentially still ranging from 0.1% to 1% by weight, with respect to the total weight of the composition (B).

### Oxidation Dyes

The oxidation dyes are generally chosen from one or more oxidation bases, optionally combined with one or more coupling agents (also known as couplers).

### Oxidation Bases

The composition (B) can optionally comprise one or more oxidation bases advantageously chosen from those conventionally used in the dyeing of keratin fibres.

By way of example, the oxidation bases are chosen from para-phenylenediamines, bis(phenyl)alkylenediamines, para-aminophenols, ortho-aminophenols and heterocyclic bases, and the corresponding addition salts.

The para-phenylenediamines which may be mentioned include, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-p-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-p-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-p-hydroxyethylamino-5-aminotoluene and 3-hydroxy-1-(4′-aminophenyl)pyrrolidine and the corresponding addition salts with an acid.

Preference is in particular given, among the abovementioned para-phenylenediamines, to para-phenylenediamine, para-toluenediamine, 2-isopropyl-para-phenylenediamine, 2-β-hydroxyethyl-para-phenylenediamine, 2-p-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the corresponding addition salts with an acid.

The bis(phenyl)alkylenediamines which may be mentioned include, for example, N,N′-bis(β-hydroxyethyl)-N,N′-bis(4′-aminophenyl)-1,3-diaminopropanol, N,N′-bis(p-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane and the corresponding addition salts.

The para-aminophenols which are mentioned include, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol and the corresponding addition salts with an acid.

The ortho-aminophenols which may be mentioned include, for example, 2-aminophenol, 2-amino-5-methylphenol, 2-amino-6-methylphenol and 5-acetamido-2-aminophenol and the corresponding addition salts.

The heterocyclic bases which may be mentioned include, for example, pyridine, pyrimidine and pyrazole derivatives.

The pyridine derivatives which may be mentioned include the compounds for example described in Patents GB 1 026 978 and GB 1 153 196, for example 2,5-diaminopyridine, 2-(4-methoxyphenyl)amino-3-aminopyridine and 3,4-diaminopyridine and the corresponding addition salts.

Other pyridine oxidation bases which are of use in the present invention are the 3-aminopyrazolo[1,5-a]pyridine oxidation bases or the corresponding addition salts described, for example, in Patent Application FR 2 801 308. Examples which may be mentioned comprise pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-p-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases which are of use in the present invention are chosen from 3-aminopyrazolo[1,5-a]pyridines which are preferably substituted on carbon atom 2 by:
a) a (di)(C₁-C₆)(alkyl)amino group, it being possible for said alkyl group to be substituted by at least one hydroxyl, amino or imidazolium group;
b) an optionally cationic 5- to 7-membered heterocycloalkyl group containing from 1 to 3 heteroatoms, optionally substituted by one or more (C₁-C₆)alkyl groups, such as a di(C₁-C₄)alkylpiperazinium group; or
c) a (C₁-C₆)alkoxy group optionally substituted by one or more hydroxyl groups, such as a β-hydroxyalkoxy group, and the corresponding addition salts.

The pyrimidine derivatives which may be mentioned include the compounds described, for example, in Patents DE 2359399, JP 88-169571, JP 05-63124 and EP 0 770 375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

The pyrazole derivatives which may be mentioned include the compounds described in Patents DE 3843892 and DE 4133957 and Patent Applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and the corresponding addition salts. Use may also be made of 4,5-diamino-1-(β-methoxyethyl)pyrazole.

A 4,5-diaminopyrazole will preferably be used and more preferentially still 4,5-diamino-1-(β-hydroxyethyl)pyrazole and/or a corresponding salt.

The pyrazole derivatives which may also be mentioned comprise diamino-N,N-dihydropyrazolopyrazolones and in particular those described in Patent Application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di(2-hydroxyethyl)-1,2-dihydropyrazol-3-one, 2-amino-3-(2-hydroxyethyl)amino-6,7-dihydro-1 H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or a corresponding salt.

### Coupling Agents

The composition (B) can optionally comprise one or more coupling agents advantageously chosen from those conventionally used in the dyeing of keratin fibres. Mention may in particular be made, among these coupling agents, of meta-phenylenediamines, meta-aminophenols, meta-diphenols, naphthalene-based coupling agents and heterocyclic coupling agents, and also the corresponding addition salts. Mention may be made, for example, of 1,3-dihydroxybenzene, 1,3-dihydroxy-2-methylbenzene, 4-chloro-1,3-dihydroxybenzene, 2,4-diamino-1-(p-hydroxyethyloxy)benzene, 2-amino-4-(p-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-p-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(p-hydroxyethyl)amino-3,4-methylenedioxybenzene, 2,6-bis(p-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, 2-methyl-5-aminophenol, 5-N-(p-hydroxyethyl)amino-2-methylphenol, 3-aminophenol and 3-amino-2-chloro-6-methylphenol, the corresponding addition salts with an acid and the corresponding mixtures.

In general, the addition salts of oxidation bases and of coupling agents which can be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

The oxidation base(s) each advantageously represent(s) from 0.001% to 10% by weight, with respect to the total weight of the composition (B), and preferably from 0.005% to 5% by weight, with respect to the total weight of the composition (B).

The coupling agent(s), if they are present, each advantageously represent(s) from 0.001% to 10% by weight, with respect to the total weight of the composition (B), and preferably from 0.005% to 5% by weight, with respect to the total weight of the composition (B).

### Direct Dyes

The composition (B) can additionally comprise one or more direct dyes.

The synthetic direct dyes which can be used are, for example, chosen from those conventionally used for direct dyeing, and among which mention may be made of all the aromatic and/or non-aromatic dyes which are commonly used, such as nitrobenzene, azo, hydrazono, nitro(hetero)aryl, tri(hetero)arylmethane, (poly)methine, carbonyl, azine, porphyrin, metalloporphyrin, quinone and in particular anthraquinone, indoamine and phthalocyanine direct dyes and their mixtures.

Mention may be made, among the nitrobenzene direct dyes, of: 1,4-diamino-2-nitrobenzene, 1-amino-2-nitro-4-β-hydroxyethylaminobenzene, 1-amino-2-nitro-4-bis(p-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl)(β-hydroxyethyl)aminobenzene, 1-amino-3-methyl-4-p-hydroxyethylamino-6-nitrobenzene, 1-amino-2-nitro-4-p-hydroxyethylamino-5-chlorobenzene, 1,2-diamino-4-nitrobenzene, 1-amino-2-p-hydroxyethylamino-5-nitrobenzene, 1,2-bis(β-hydroxyethylamino)-4-nitrobenzene, 1-amino-2-tris(hydroxymethyl)methylamino-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-p-hydroxyethyloxy-2-p-hydroxyethylamino-5-nitrobenzene, 1-methoxy-2-p-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-p-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-p-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-bis(β-hydroxyethyl)amino-3-nitrobenzene, 1-p-hydroxyethylamino-2-nitrobenzene, 1-hydroxy-4-p-hydroxyethylamino-3-nitrobenzene.

Mention may be made, among the azo direct dyes, of: Basic Red 51, Basic Orange 31, Disperse Red 17, Acid Yellow 9, Acid Black 1, Basic Red 22, Basic Red 76, Basic Yellow 57, Acid Yellow 36, Acid Orange 7, Acid Red 33, Acid Red 35, Acid Yellow 23, Acid Orange 24, Disperse Black 9, Basic Brown 16, Basic Brown 17.

Mention may be made, among the hydrazono direct dyes, of: Basic Yellow 87.

Mention may be made, among the nitroaryl direct dyes, of: HC Blue 2, HC Yellow 2, HC Red 3, 4-hydroxypropylamino-3-nitrophenol, N,N′-bis(2-hydroxyethyl)-2-nitrophenylenediamine.

Mention may be made, among the triarylmethane direct dyes, of: Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), Acid Blue 1; Acid Blue 3; Acid Blue 7, Acid Blue 9; Acid Violet 49; Acid Green 3; Acid Green 5; Acid Green 50.

Mention may be made, among the quinone direct dyes, of: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino)anthraquinone, Acid Blue 25, Acid Blue 43, Acid Blue 78, Acid Blue 129, Acid Blue 138, Acid Blue 140, Acid Blue 251, Acid Green 25, Acid Green 41, Acid Violet 42, Mordant Red 3, Acid Black 48, HC Blue 16.

Mention may be made, among the azine direct dyes, of: Basic Blue 17, Basic Red 2.

Mention may be made, among the indoamine direct dyes, of: 2-β-hydroxyethylamino-5-[bis(β-4′-hydroxyethyl)amino]anilino-1,4-benzoquinone, 2-β-hydroxyethylamino-5-(2′-methoxy-4′-amino)anilino-1,4-benzoquinone, 3-N-(2′-chloro-4′-hydroxy)phenylacetylamino-6-methoxy-1,4-benzoquinoneimine, 3-N-(3′-chloro-4′-methylamino)phenylureido-6-methyl-1,4-benzoquinoneimine, 3-[4′-N-(ethylcarbamylmethyl)amino]phenylureido-6-methyl-1,4-benzoquinoneimine.

The natural direct dyes which can be used are, for example, chosen from lawsone, juglone, indigo, leucoindigo, indirubin, isatin, hennotannic acid, alizarin, carthamin, morin, purpurin, carminic acid, kermesic acid, laccaic acid, purpurogallin, protocatechualdehyde, curcumin, spinulosin, apigenidin, orceins, carotenoids, betanin, chlorophylls, chlorophyllins, monascus, polyphenols or ortho-diphenols.

Mention may be made, among the ortho-diphenols of use according to the invention, of: catechin, quercetin, brazilin, haematein, haematoxylin, chlorogenic acid, caffeic acid, gallic acid, L-DOPA, cyanidin, (−)-epicatechin, (−)-epigallocatechin, (−)-epigallocatechin 3-gallate (EGCG), isoquercetin, pomiferin, esculetin, 6,7-dihydroxy-3-(3-hydroxy-2,4-dimethoxyphenyl)coumarin, santalin A and B, mangiferin, butein, maritimetin, sulfuretin, robtein, betanidin, pericampylinone A, theaflavin, proanthocyanidin A2, proanthocyanidin B2, proanthocyanidin C1, procyanidins DP 4-8, tannic acid, purpurogallin, 5,6-dihydroxy-2-methyl-1,4-naphthoquinone, alizarin, wedelolactone and the natural extracts containing them.

The direct dye(s) can be present in the the composition (B) in a total content ranging from 0.001% to 5% by weight, preferably from 0.01% to 3% by weight, more preferentially from 0.1% to 1% by weight, more preferentially still from 0.1% to 0.8% by weight, with respect to the total weight of the composition (B).

According to a preferred embodiment, the composition (B) comprises a total content of colouring agents of less than 0.1% by weight, preferably of less than 0.01% by weight, more preferentially of less than 0.001% by weight, with respect to the total weight of the composition (B).

According to a more preferred embodiment, the composition (B) is devoid of colouring agents.

### pH of the Composition (B)

The composition (B) preferably exhibits a pH of less than or equal to 11, preferably of less than or equal to 10.5, preferably of less than or equal to 10.

The pH of the composition (B) can vary from 8 to 11, preferably from 8 to 10.5, more preferentially from 8 to 10.

According to a particularly preferred embodiment, the pH of the composition (B) according to the invention varies from 8.3 to 10.

The composition (B) can additionally comprise one or more compounds D as defined above. The various characteristics of the compounds D described above apply also to the compounds D which can be included in the composition (B).

The compound(s) D are present in the composition (B) preferably in a total content by weight ranging from 1% to 20% by weight, more preferentially ranging from 2% to 15% by weight, more preferentially still ranging from 5% to 12% by weight, with respect to the total weight of the composition (B).

Compositions (A), (B) and/or (C)

### Basifying Agents

The compositions (A) and/or (B) and/or (C) can additionally comprise one or more basifying agents other than the carbonates, bicarbonates and silicates as defined above.

The basifying agent(s) can be inorganic or organic. They can be chosen from i) aqueous ammonia, ii) alkanolamines, such as mono-, di- and triethanolamine and also their derivatives, iii) oxyethylenated and/or oxypropylenated ethylenediamines, iv) inorganic or organic hydroxides, v) amino acids, preferably basic amino acids, such as arginine, lysine, ornithine, citrulline and histidine, and vi) the compounds of following formula (II):

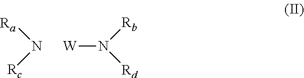

in which:
W is a divalent (C₁-C₈)alkylene group, preferably a propylene group, optionally substituted in particular by a hydroxyl group or a C₁-C₄ alkyl radical;
Rₐ, R_{b}, R_{c} and R_{d}, which can be identical or different, represent a hydrogen atom or a C₁-C₄ alkyl or C₁-C₄ hydroxyalkyl radical;
   vii) and their mixtures.

The inorganic or organic hydroxides are preferably chosen from i) alkali metal hydroxides, such as sodium hydroxide or potassium hydroxide, ii) alkaline earth metal hydroxides, iii) transition metal hydroxides, such as hydroxides of metals from Groups III, IV, V and VI, iv) hydroxides of lanthanides or of actinides.

When they are present, the basifying agent(s) other than the carbonates, bicarbonates and silicates as defined above represent preferably from 0.001% to 20% by weight, more particularly from 0.005% to 16% by weight, with respect to the total weight of the composition (A) or (B) or (C).

According to a preferred embodiment, the compositions (A) and/or (B) and/or (C) do not comprise a basifying agent chosen from aqueous ammonia and/or alkanolamines.

### Acidifying Agents

The compositions (A) and/or (B) and/or (C) can additionally comprise one or more acidifying agents.

Mention may be made, among the acidifying agents, by way of example, of inorganic acids, such as, for example, hydrochloric acid, (ortho)phosphoric acid, boric acid, nitric acid or sulfuric acid, or organic acids, such as, for example, compounds comprising at least one carboxylic acid function, such as acetic acid, tartaric acid, citric acid or lactic acid, one sulfonic acid function, one phosphonic acid function or one phosphoric acid function.

### Sequestrants

The compositions (A) and/or (B) and/or (C) can optionally comprise one or more sequestrants. Mention may be made, by way of examples of sequestrants which can be used in the present invention, of N,N-bis(carboxymethyl)-L-glutamic acid and tetrasodium N,N-bis(carboxymethyl)-L-glutamate.

Other Characteristics of the Compositions (A), (B) and/or (C)

The compositions (A) and/or (B) and/or (C) preferably comprise water in a content ranging from 5% to 99% by weight, more preferentially ranging from 5% to 80% by weight, with respect to the total weight of the composition.

The compositions (A) and/or (B) and/or (C) can additionally comprise at least one organic solvent.

The term “organic solvent” is understood to mean an organic substance which is capable of dissolving another substance without chemically modifying it.

Mention may be made, as organic solvent, for example, of lower C₂-C₄ alkanols, such as ethanol and isopropanol; polyols and polyol ethers, such as 2-butoxyethanol, propylene glycol, propylene glycol monomethyl ether, diethylene glycol monoethyl ether or diethylene glycol monomethyl ether, and also aromatic alcohols, such as benzyl alcohol or phenoxyethanol, and their mixtures.

The organic solvents are present in proportions ranging preferably from 0.1% to 40% by weight, more preferentially from 1% to 30% by weight, more preferentially still from 1% to 25% by weight, with respect to the total weight of the composition (A) and/or (B) and/or (C).

The compositions (A) and/or (B) and/or (C) can be provided in liquid form, in the form of a serum, in thickened form, in particular a gel, a cream, a wax or a paste, or in foam form.

The compositions (A) and/or (B) and/or (C) can additionally comprise one or more additional compounds chosen from non-ionic, anionic, cationic or amphoteric surfactants, associative or non-associative, cationic, anionic, non-ionic or zwitterionic, thickening polymers of natural or synthetic origin, silicones in the form of oils, gums or resins or non-silicone vegetable, mineral or synthetic oils, UV-screening agents, fillers, such as pearlescent agents and metal oxides, such as titanium dioxides, clays, fragrances, peptizing agents, vitamins and preservatives.

### Additional Features of the Treatment Method

According to a preferred embodiment, the method according to the present invention is a method for lightening keratin fibres.

Preferably, the keratin fibres treated according to the method of the present invention are dark keratin fibres.

The term “dark keratin fibres” is understood to mean keratin fibres, the tone depth of which is less than or equal to 6 (dark blond), preferably less than or equal to 4 (chestnut).

The process according to the present invention makes it possible to obtain a lightening of keratin fibres characterized by a value of b* which is lower, preferably lower by 10%, more preferentially lower by 15%, than the value of b* measured at the same level of intensity L* on keratin fibres which have been lightened using a composition comprising one or more persulfates, the values of b* and L* being measured in the CIE L*a*b* system.

Preferably, the values of b* and L* are measured according to the method of evaluation of the colour described in the examples.

### Step a)

During step a), the composition (A) is applied to dry or wet keratin fibres, preferably to dry keratin fibres.

The composition (A) is applied to the keratin fibres in an amount preferably ranging from 0.1 g to 20 g, more preferentially ranging from 0.5 g to 10 g, of composition (A) per gram of keratin fibres.

The composition (A) is left on the keratin fibres for a period of time preferably ranging from 30 seconds to 30 minutes. By way of example, the composition (A) is left on the keratin fibres for 5 minutes.

The working temperature of step a) is conventionally between ambient temperature (between 15° C. and 25° C.) and 70° C.

According to a preferred embodiment, step a) is carried out at ambient temperature (between 15° C. and 25° C.).

The composition (A) can be left on the fibres under an occlusive system. Mention may be made, as non-limiting example of an occlusive system, of an occlusive system of wrapper type made of aluminium or plastic film or hair cap type, with or without holes.

On conclusion of step a), the keratin fibres are optionally rinsed with water or optionally washed with a shampoo and then rinsed with water, before being optionally dried or left to dry naturally.

The drying step can be carried out using absorbent paper, a hairdryer or a styling hood.

Preferably, the keratin fibres are neither rinsed with water nor washed with a shampoo on conclusion of step a).

Preferably, the keratin fibres are dried or left to dry naturally on conclusion of step a).

During the implementation of the method according to the present invention, step a) can be repeated several times.

### Step b)

The composition (B) is applied to the keratin fibres in an amount preferably ranging from 0.1 g to 20 g of composition per gram of keratin fibres.

The composition (B) is left on the keratin fibres for a period of time preferably ranging from 1 minute to 1 hour, more preferentially ranging from 5 minutes to 60 minutes. By way of example, the composition (B) is left on the keratin fibres for 50 minutes.

The working temperature of step b) is conventionally between ambient temperature (between 15° C. and 25° C.) and 80° C., preferably between ambient temperature and 60° C. According to a preferred embodiment, step b) is carried out at 33° C.

The composition (B) can be left on the fibres under an occlusive system. Mention may be made, as non-limiting example of an occlusive system, of an occlusive system of wrapper type made of aluminium or plastic film or hair cap type, with or without holes.

On conclusion of step b), the keratin fibres are optionally rinsed with water or optionally washed with a shampoo and then rinsed with water, before being optionally dried or left to dry naturally.

The drying step can be carried out using absorbent paper, a hairdryer or a styling hood.

The composition (B) employed in the method according to the present invention is preferably prepared by mixing at least two compositions. Preferably, the mixing of said at least two compositions is carried out extemporaneously, before the application of the composition to the keratin fibres.

According to a preferred embodiment, the composition (B) results from the mixing:
of a composition (B1) comprising:
one or more chemical oxidizing agents chosen from hydrogen peroxide, hydrogen peroxide-generating systems other than peroxygenated salts and their mixtures as defined above, preferably hydrogen peroxide; and
of a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
optionally one or more colouring agents, preferably chosen from direct dyes, oxidation dyes and their mixtures; and
optionally of a composition (B3) comprising:
one or more colouring agents, preferably chosen from direct dyes, oxidation dyes and their mixtures.

According to a preferred embodiment, the method according to the present invention is a method for lightening keratin fibres in which the composition (B) results from the mixing:
of a composition (B1) comprising:
one or more chemical oxidizing agents chosen from hydrogen peroxide, hydrogen peroxide-generating systems other than peroxygenated salts and their mixtures as defined above, preferably hydrogen peroxide; and
of a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above.

According to another preferred embodiment, the method according to the present invention is a method for simultaneous bleaching and dyeing of keratin fibres in which the composition (B) results from the mixing:
of a composition (B1) comprising:
one or more chemical oxidizing agents chosen from hydrogen peroxide, hydrogen peroxide-generating systems other than peroxygenated salts and their mixtures as defined above, preferably hydrogen peroxide; and
of a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
one or more colouring agents, preferably chosen from direct dyes, oxidation dyes and their mixtures.

According to another preferred embodiment, the method according to the present invention is a method for simultaneous bleaching and dyeing of keratin fibres in which the composition (B) results from the mixing:
of a composition (B1) comprising:
one or more chemical oxidizing agents chosen from hydrogen peroxide, hydrogen peroxide-generating systems other than peroxygenated salts and their mixtures as defined above, preferably hydrogen peroxide; and
of a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
of a composition (B3) comprising one or more colouring agents, preferably chosen from direct dyes, oxidation dyes and their mixtures.

Preferably, at least one of the compositions (B1) or (B2) or at least one of the three compositions (B1) or (B2) or (B3) is aqueous. More preferentially, the composition (B1) is aqueous.

According to a specific embodiment, the composition (B2) is anhydrous.

According to a specific embodiment, the composition (B1) is aqueous, the composition (B2) is anhydrous and the composition (B3), when it is present, is aqueous.

The term “aqueous composition” is understood to mean a composition comprising at least 2% by weight of water, preferably at least 5% by weight of water, more preferentially at least 10% by weight of water and more advantageously still more than 20% by weight of water.

The term “anhydrous composition” is understood to mean a composition containing less than 2% by weight of water, preferably less than 0.5% by weight of water, more preferentially devoid of water. If appropriate, such small amounts of water can in particular be introduced by ingredients of the composition which may contain residual amounts thereof.

### Step c)

During step c), the composition (C) is applied to dry or wet keratin fibres, preferably to wet keratin fibres.

The composition (C) is applied to the keratin fibres in an amount preferably ranging from 0.01 g to 5 g, more preferentially ranging from 0.05 g to 5 g, of composition (C) per gram of keratin fibres.

The composition (C) is left on the keratin fibres for a period of time preferably ranging from 30 seconds to 30 minutes, more preferentially ranging from 2 minutes to 10 minutes.

The working temperature of step c) is conventionally between ambient temperature (between 15° C. and 25° C.) and 70° C.

According to a preferred embodiment, step c) is carried out at ambient temperature (between 15° C. and 25° C.).

The composition (C) can be left on the fibres under an occlusive system. Mention may be made, as non-limiting example of an occlusive system, of an occlusive system of wrapper type made of aluminium or plastic film or hair cap type, with or without holes.

On conclusion of step c), the keratin fibres are optionally rinsed with water or optionally washed with a shampoo and then rinsed with water, before being optionally dried or left to dry naturally.

The drying step can be carried out using absorbent paper, a hairdryer or a styling hood.

Preferably, the keratin fibres are rinsed with water on conclusion of step a).

During the implementation of the method according to the present invention, step c) can be repeated several times. By way of example, step c) can be repeated from 1 to 5 times with preferably a drying step between each application of the composition (C).

According to one embodiment, the composition (C) is provided in the form of a shampoo. According to this embodiment, on conclusion of step c), the keratin fibres are preferably rinsed with water and optionally treated using a composition of conditioner, lotion or mask type, before being optionally dried or left to dry naturally.

According to another preferred embodiment, composition (C) is in the form of a hair conditioner composition, e.g. a cream rinse, a lotion or a mask.

According to this other embodiment, the method comprises, between step b) and c), a step of washing the keratin fibres with a shampoo, followed by rinsing with water.

According to this other embodiment, on conclusion of step c), the keratin fibres are preferably rinsed with water, before being optionally dried or left to dry naturally.

### Composition (B)

According to a second aspect, a subject-matter of the present invention is a composition (B) as defined above comprising one or more compounds chosen from chitosans, vitamins B, vitamin C, vanillin, their salts, their solvates and their mixtures, preferably chosen from chitosans, vitamins B, their salts, their solvates and their mixtures.

The compound(s) chosen from chitosans, vitamins B, vitamin C, vanillin, their salts, their solvates and their mixtures are present in the composition (B) in a total content by weight preferably ranging from 1% to 20% by weight, more preferentially ranging from 2% to 15% by weight, more preferentially still ranging from 2% to 12% by weight, with respect to the total weight of the composition (B).

### Use

According to a third aspect, a subject-matter of the present invention is the use of the composition(s) (A) and/or (C) as defined above for reducing the damage to keratin fibres treated by a method for the treatment of keratin fibres employing the composition (B) as defined above.

According to a preferred embodiment, a subject-matter of the present invention is the use of the composition(s) (A) and/or (C) as defined above for protecting and/or repairing the keratin fibres treated by a method for the treatment of keratin fibres employing the composition (B) as defined above.

According to a more preferred embodiment, a subject-matter of the present invention is the use of the composition(s) (A) and/or (C) as defined above for reducing the breakage of the keratin fibres treated by a method for the treatment of keratin fibres employing the composition (B) as defined above.

### Multicompartment Device (Kit)

According to a fourth aspect, a subject-matter of the present invention is a multicompartment device (kit) comprising:
a first compartment including a composition (B1) comprising:
one or more chemical oxidizing agents chosen from hydrogen peroxide, hydrogen peroxide-generating systems other than peroxygenated salts and their mixtures as defined above, preferably hydrogen peroxide; and
a second compartment including a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
optionally one or more colouring agents, preferably chosen from direct dyes, oxidation dyes and their mixtures; and
optionally one or more compounds D as defined above; and
a third compartment including a composition (A) or (C) as defined above, it being possible for the composition (A) or (C) to optionally comprise one or more colouring agents, preferably chosen from direct dyes, oxidation dyes and their mixtures; and
optionally a fourth compartment including a composition (B3) comprising one or more colouring agents, preferably chosen from direct dyes, oxidation dyes and their mixtures.

According to a specific embodiment, the multicompartment device (kit) comprises:
a first compartment including a composition (B1) comprising:
one or more chemical oxidizing agents chosen from hydrogen peroxide, hydrogen peroxide-generating systems other than peroxygenated salts and their mixtures as defined above, preferably hydrogen peroxide; and
a second compartment including a composition (B2) comprising:
one or more compounds chosen from carbonates, carbonate-generating systems, bicarbonates, bicarbonate-generating systems and their mixtures as defined above, preferably chosen from carbonates, bicarbonates and their mixtures as defined above; and
one or more silicates as defined above; and
optionally one or more colouring agents, preferably chosen from direct dyes, oxidation dyes and their mixtures; and
optionally one or more compounds D as defined above; and
a third compartment including a composition (A) as defined above, it being possible for the composition (A) to optionally comprise one or more colouring agents, preferably chosen from direct dyes, oxidation dyes and their mixtures; and
a fourth compartment including a composition (C) as defined above, it being possible for the composition (C) to optionally comprise one or more colouring agents, preferably chosen from direct dyes, oxidation dyes and their mixtures; and
optionally a fifth compartment including a composition (B3) comprising one or more colouring agents, preferably chosen from direct dyes, oxidation dyes and their mixtures.

### EXAMPLES

The examples which follow make possible a better understanding of the invention without, however, exhibiting a limiting nature. In the examples which follow, unless otherwise indicated, all the amounts are shown as percentages by weight, with respect to the total weight of the composition.

### Examples 1 to 14 and 14a: Method for the Treatment of Keratin Fibres with Pretreatment Step a)

The following pretreatment compositions A1 to A9 and also the bleaching compositions Bᵤ to B_{z} were prepared, then applied according to the application protocol described below and evaluated according to the evaluation protocols described below:

### Pretreatment Compositions

TABLE 1

| Ingredients | A1 | A2 | A3 | A4 | A5 |
|---|---|---|---|---|---|
| Pyridoxine | 10 | — | — | — | — |
| Hydrochloride | | | | | |
| CAS: [58-56-0] | | | | | |
| Levulinic Acid | — | 5 | — | — | — |
| CAS: [123-76-2] | | | | | |
| L-Carnitine | — | — | 10 | — | — |
| CAS: [541-15-1] | | | | | |
| Tetrasodium | — | — | — | 20 | — |
| Glutamate Diacetate | | | | | |
| CAS: [51981-21-6] | | | | | |
| Thiamine | — | — | — | — | 10 |
| Hydrochloride | | | | | |
| CAS: [67-03-8] | | | | | |
| Sodium Hydroxide | q.s. | q.s. | q.s. | q.s. | q.s. |
| or Hydrochloric | pH = | pH = | pH = | pH = | pH = |
| Acid | 9 | 8.4 | 9.3 | 8.9 | 8.7 |
| Water | q.s. | q.s. | q.s. | q.s. | q.s. |
| | for 100 | for 100 | for 100 | for 100 | for 100 |

TABLE 2

| Ingredients | A6 | A7 | A8 | A9 |
|---|---|---|---|---|
| Oleic Acid | 10 | — | — | — |
| CAS: [112-80-1] | | | | |
| Citric Acid | — | 10 | — | — |
| CAS: [77-92-9] | | | | |
| Glycine | — | — | 20 | — |
| CAS: [56-40-6] | | | | |
| Chitosan | — | — | — | 5 |
| MW: 5 kDa | | | | |
| Sodium Hydroxide | q.s. | q.s. | q.s. | q.s. |
| or Hydrochloric | pH = 9.1 | pH = 9 | pH = 8.8 | pH = 4.0 |
| Acid | | | | |
| Water | q.s. | q.s. | q.s. | q.s. |
| | for 100 | for 100 | for 100 | for 100 |

### Bleaching Compositions

TABLE 3

| Ingredients | Bᵤ | Bᵥ | B_{w} | Bₓ |
|---|---|---|---|---|
| Oxidizing Cream | 66 | 66 | 66 | 66 |
| Blond Studio 40 Vol. | (i.e. 7.9 | (i.e. 7.9 | (i.e. 7.9 | (i.e. 7.9 |
| L'Oréal Professionnel | of H₂O₂) | of H₂O₂) | of H₂O₂) | of H₂O₂) |
| (12% of H₂O₂) | | | | |
| Sodium Silicate | 17 | 11 | 11 | 11 |
| CAS: [1344-09-8] | | | | |
| Ammonium bicarbonate | 17 | 11 | 11 | 11 |
| Thiamine | — | — | 7.7 | — |
| Hydrochloride | | | | |
| CAS: [67-03-8] | | | | |
| Pyridoxine | — | — | — | 7.7 |
| Hydrochloride | | | | |
| CAS: [58-56-0] | | | | |
| Water | — | q.s. | q.s. | q.s. |
| | | for 100 | for 100 | for 100 |

TABLE 4

| Ingredients | B_{y} | B_{z} |
|---|---|---|
| Oxidizing Cream | 66 | 66 |
| Blond Studio 40 Vol. | (i.e. 7.9 | (i.e. 7.9 |
| L'Oréal Professionnel | of H₂O₂) | of H₂O₂) |
| (12% of H₂O₂) | | |
| Sodium Silicate | 11 | 11 |
| CAS: [1344-09-8] | | |
| Ammonium Bicarbonate | 11 | 11 |
| L-Carnitine | 7.7 | — |
| CAS: [541-15-1] | | |
| Water | q.s. for 100 | q.s. for 100 |

### Application Protocol

### Step a): Pretreatment

The compositions A1 to A9 were applied to locks of hair of HT4 Caucasian type, according to the bath ratios (g of composition/g of hair) shown in the table below. The hair was massaged with the fingers, making circular movements along the entire length of the lock (5 times), and the locks were then left for 5 minutes. The locks were subsequently wrung out and then dried with a hairdryer.

### Step b): Bleaching

The compositions Bᵤ to B_{z} were applied to the locks of hair treated during step a) according to the bath ratio (g of composition/g of hair) shown in the table below. The locks were subsequently placed on a hot plate at 33° C. (temperature representative of the temperature of the individual hair on the head) for 25 minutes, then turned over and left for another 25 minutes on the hot plate. The locks were subsequently rinsed with tap water for 30 seconds, wrung out, shampooed with a DOP shampoo, wrung out and then dried with a hairdryer.

The locks were subsequently evaluated as regards the colour and the integrity according to the evaluation protocols described below.

### Bath Ratios

TABLE 5

| | Step a) | | Step b) | |
|---|---|---|---|---|
| | | Bath ratio of | | Bath ratio of |
| | Pretreatment | the pretreatment | Bleaching | the bleaching |
| Example | composition | composition | composition | composition |
| 1 | A1 | 10 | Bᵤ | 5 |
| 2 | A2 | 3 | Bᵤ | 5 |
| 3 | A3 | 5 | Bᵤ | 5 |
| 4 | A4 | 0.5 | Bᵤ | 5 |
| 5 | A5 | 2 | Bᵤ | 5 |
| 6 | A6 | 3 | Bᵤ | 5 |
| 7 | A7 | 3 | Bᵤ | 5 |
| 8 | A8 | 2 | Bᵤ | 5 |
| 9 | A4 | 0.5 | Bᵥ | 10 |
| 10 | A6 | 3 | Bᵥ | 10 |
| 11 | A9 | 5 | Bᵥ | 10 |
| 12 | A5 | 2 | B_{w} | 10 |
| 13 | A1 | 10 | Bₓ | 10 |
| 14 | A3 | 5 | B_{y} | 10 |
| 14a | A9 | 2 | Bᵥ | 10 |

### Method of Evaluation of the Colour

The colour of the locks was evaluated in the CIE L* a* b* system, using a Minolta Spectrophotometer CM2600D colorimeter (illuminant D65).

In this L* a* b* system, L* represents the intensity of the colour, a* indicates the shade of the colour on the green/red colour axis and b* indicates the shade of colour on the blue/yellow colour axis. The higher the value of L*, the lighter the colour. The higher the value of a*, the redder the colour, and the higher the value of b*, the yellower the colour.

### Method of Evaluation of the Integrity of the Individual Hair

This test consists in pulling on the wet individual hair to see if the individual hair breaks, is irreversibly deformed or is deformed and then returns to its initial shape and length. The test is commonly used by hairstylists to evaluate the integrity of their clients' hair. The test consists in taking 6 fibres of hair, soaking them in water for 30 seconds, then grasping them in the hands, moving the fingers and thumb of each hand apart so that, between the hands, there are 3 centimetres of fibres, and then pulling so that the fibres are deformed by an additional 1 cm. The fibres are held for 3 seconds then the tension is gently released. The fibres are placed horizontally on white paper for 5 minutes to dry and are then evaluated.

In the laboratory, a ruler is used to ensure that the distances are respected. The test is repeated 5 times, i.e. a total of 30 fibres.

The integrity performance of the individual hair is classified by the following rules:

If the sum of the broken and irreversibly deformed fibres is equal to or greater than 10, the individual hair has a very poor integrity.

If the sum of the broken and irreversibly deformed fibres is between 6 and 9 inclusive, the individual hair has a poor integrity.

If the sum of the broken and irreversibly deformed fibres is between 2 and 5 inclusive, the individual hair has a good integrity.

If the sum of the broken and irreversibly deformed fibres is 1 or less, the individual hair has a very good integrity.

### Colorimetric Measurements

The results of the colorimetric measurements are summarized in the following table:

TABLE 6

| Example | b* |
|---|---|
| 1 | 21.3 |
| 2 | 20.2 |
| 3 | 21.1 |
| 4 | 21.3 |
| 5 | 18.7 |
| 6 | 17.4 |
| 7 | 17.6 |
| 8 | 20.2 |
| 9 | 22.2 |
| 10 | 21.4 |
| 11 | 19.4 |
| 12 | 16.1 |
| 13 | 23.9 |
| 14 | 20.9 |
| 14a | 21.8 |

The method according to the invention makes it possible to obtain colour shades characterized by relatively low values of b* and thus a less yellow and more natural result.

### Measurements of the Integrity of the Individual Hair

The results of the integrity measurements are summarized in the following table:

TABLE 7

| | Number of | Number of fibres | |
|---|---|---|---|
| | fibres which | which have been | Comments on |
| Example | have broken | irreversibly deformed | the integrity |
| 1 | 0 | 1 | Very good |
| 2 | 0 | 1 | Very good |
| 3 | 0 | 1 | Very good |
| 4 | 0 | 1 | Very good |
| 5 | 0 | 0 | Very good |
| 6 | 0 | 1 | Very good |
| 7 | 0 | 1 | Very good |
| 8 | 0 | 1 | Very good |
| 9 | 0 | 2 | Good |
| 10 | 0 | 1 | Very good |
| 11 | 1 | 4 | Good |
| 12 | 0 | 0 | Very good |
| 13 | 0 | 1 | Very good |
| 14 | 0 | 1 | Very good |
| 14a | 2 | 1 | Good |

The method according to the invention is respectful of the integrity of the fibre and makes it possible to protect the hair, in particular from breakage or from deformation.

### Examples 15 to 25: Method for the Treatment of Keratin Fibres with a Post-Treatment Step b)

The following post-treatment compositions C1 to C11 and also the bleaching composition Bᵥ described in Examples 1 to 14 and 14a were prepared and then applied according to the application protocol described below and evaluated according to the protocol for evaluation of the colour described in Examples 1 to 14 and 14a and according to the evaluation protocols described below. Composition C11 was also evaluated according to the method of evaluation of the integrity of the individual hair described in Examples 1 to 14 and 14a.

TABLE 8

| Ingredients | C1 | C2 | C3 | C4 |
|---|---|---|---|---|
| Pyridoxine | 6.1 | — | — | — |
| Hydrochloride | | | | |
| CAS: [58-56-0] | | | | |
| Levulinic Acid | — | 3.5 | — | — |
| CAS: [123-76-2] | | | | |
| L-Carnitine | — | — | 4.8 | — |
| CAS: [541-15-1] | | | | |
| Tetrasodium | — | — | — | 10.5 |
| Glutamate Diacetate | | | | |
| CAS: [51981-21-6] | | | | |
| Sodium Hydroxide | q.s. | q.s. | q.s. | q.s. |
| or Hydrochloric | pH = 4 | pH = 4 | pH = 4 | pH = 4 |
| Acid | | | | |
| Water | q.s. | q.s. | q.s. | q.s. |
| | for 100 | for 100 | for 100 | for 100 |

TABLE 9

| Ingredients | C5 | C6 | C7 | C8 |
|---|---|---|---|---|
| Thiamine | 10.1 | — | — | — |
| Hydrochloride | | | | |
| CAS: [67-03-8] | | | | |
| Oleic Acid | — | 8.5 | — | — |
| CAS: [112-80-1] | | | | |
| Citric Acid | — | — | 5.7 | — |
| CAS: [77-92-9] | | | | |
| Glycine | — | — | — | 2.2 |
| CAS: [56-40-6] | | | | |
| Sodium Hydroxide | q.s. | q.s. | q.s. | q.s. |
| or Hydrochloric | pH = 4 | pH = 4 | pH = 4 | pH = 4 |
| Acid | | | | |
| Water | q.s. | q.s. | q.s. | q.s. |
| | for 100 | for 100 | for 100 | for 100 |

TABLE 10

| Ingredients | C9 | C10 | C11 |
|---|---|---|---|
| Arginine | 5.2 | — | — |
| CAS: [74-79-3] | | | |
| Citric Acid | — | 2.9 | — |
| CAS: [77-92-9] | | | |
| Glycine | — | 5.0 | — |
| CAS: [56-40-6] | | | |
| Chitosan | — | — | 5.0 |
| MW: 100 kDa | | | |
| Sodium Hydroxide | q.s. pH = 4 | q.s. pH = 4 | q.s. pH = 4 |
| or Hydrochloric | | | |
| Acid | | | |
| Water | q.s. for 100 | q.s. for 100 | q.s. for 100 |

### Application Protocol

### Step b): Bleaching

The bleaching composition Bᵥ was applied to 11 locks of hair of HT4 Caucasian type, according to a bath ratio (g of composition/g of hair) equal to 10.

The locks were subsequently placed on a hot plate at 33° C. (temperature representative of the temperature of the individual hair on the head) for 25 minutes, then turned over and left for another 25 minutes on the hot plate. The locks were subsequently rinsed with tap water for 30 seconds, wrung out, shampooed with a DOP shampoo and then wrung out.

### Step c): Post-Treatment

The compositions C1 to C11 were subsequently applied to the 11 locks of hair treated during step b) according to a bath ratio (g of composition/g of hair) equal to 2. The hair was massaged with the fingers, making circular movements along the entire length of the lock (5 times), and the locks were then left for 5 minutes. The locks were then rinsed with tap water for 30 seconds, wrung out and then dried at 60° C.

The application of the compositions C1 to C11 was repeated a further 4 times for a total of 5 applications.

### Method of Evaluation of the Repair of the Hair Fibres by Differential Scanning Calorimetry

The differential scanning calorimetry technique is known to a person skilled in the art as a method which makes it possible to quantify the strengthening of proteins in the cortex of keratin fibres (Kinetics of the changes imparted to the main structural components of human hair by thermal treatment, https://doi.org/10.1016/j.tca.2018.01.014 & F.-J. Wortmann and H. Deutz, J. Appl. Polym. Sci., 48, 137 (1993). The principle of the test is to measure the protein denaturing temperature. It is widely acknowledged that the higher the protein denaturing temperature, the better the integrity of the proteins of the cortex, which reflects the reduction in the breakage of the fibres.

The denaturing temperature is directly linked to the density of bondings of the keratin proteins present in the cortex. Thus, the lower the denaturing temperature, the lower the density of bondings of the proteins with one another, the disulfide bridges break and the cortex is damaged. A difference of 2° C. is acknowledged by a person skilled in the art as a significant modification.

The apparatus used for carrying out the measurements is a TA Instruments DSC2500 reference apparatus (temperature range: 30° C.-200° C.; heating gradient of 5° C./min). This apparatus measures the energy flow during the heating of the sample. The temperature at which the energy flow is at a maximum represents the denaturing temperature.

### Method of Evaluation of the Hair Weight Loss

The locks of hair are placed for 12 hours in a glove box having controlled hygrometry RH 45%, then weighed at ambient temperature before treatments.

Subsequent to the treatments, the dried locks are placed back in the glove box having controlled hygrometry RH 45% for 12 h and then weighed again.

The hair weight loss is thus calculated for each of the locks from the values of weights before and after treatments.

The percentage of hair weight loss is expressed by means of the following equation:

$$\% hair weight loss = 100 - \frac{{w}_{1}}{{w}_{0}} * 100$$

with:
w₀: initial weight of the lock before treatment
w₁: weight of the lock after total treatment.

### Colorimetric Measurements

The results of the colorimetric measurements are summarized in the following table:

TABLE 11

| | Post-treatment | |
|---|---|---|
| Example | composition | b* |
| 15 | C1 | 23.9 |
| 16 | C2 | 23.5 |
| 17 | C3 | 23.3 |
| 18 | C4 | 22.9 |
| 19 | C5 | 22.8 |
| 20 | C6 | 23.2 |
| 21 | C7 | 24.4 |
| 22 | C8 | 21.9 |
| 23 | C9 | 22.1 |
| 24 | C10 | 24.5 |
| 25 | C11 | 21.8 |

The method according to the invention makes it possible to obtain colour shades characterized by relatively low values of b* and thus a less yellow and more natural result.

### Measurements of the Repair of the Hair Fibres by Differential Scanning Calorimetry

The results of the measurements of the denaturing temperature of each of the locks are summarized in the following table and correspond to the mean of the measurements carried out for 6 individual hairs.

TABLE 12

| | Post-treatment | Mean denaturing |
|---|---|---|
| Example | composition | temperature (° C.) |
| 15 | C1 | 162.0 |
| 16 | C2 | 162.3 |
| 17 | C3 | 161.6 |
| 18 | C4 | 160.7 |
| 19 | C5 | 160.1 |
| 20 | C6 | 154.3 |
| 21 | C7 | 161.8 |
| 22 | C8 | 152.5 |
| 23 | C9 | 152.2 |
| 24 | C10 | 161.4 |

The method according to the invention is respectful of the integrity of the fibre and makes it possible to repair the hair.

### Measurements of Hair Weight Loss

The results of the measurements of hair weight loss are summarized in the following table:

TABLE 13

| | Post-treatment | |
|---|---|---|
| Example | composition | Hair weight loss |
| 15 | C1 | 0.0 |
| 16 | C2 | 0.6 |
| 18 | C4 | 0.4 |
| 20 | C6 | 0.0 |
| 23 | C9 | 0.5 |
| 25 | C11 | 0.0 |

The method according to the invention is respectful of the integrity of the fibre. Breakage of the individual hair is limited.

### Measurements of the Integrity of the Individual Hair

The results of the integrity measurements are summarized in the following table:

TABLE 14

| | Number of | Number of fibres | |
|---|---|---|---|
| | fibres which | which have been | Comments on |
| Example | have broken | irreversibly deformed | the integrity |
| 25 | 1 | 1 | Good |

The method according to the invention is respectful of the integrity of the fibre and makes it possible to protect the hair, in particular from breakage or from deformation.

1-27. (canceled)

28. A method for treating keratin fibers comprising:
a) optionally, applying to the keratin fibers a composition (A) comprising one or more compounds D chosen from organic acids, amino acids, chitosans, vitamins B, vitamin C, vanillin, salts thereof, solvates thereof, or mixtures of two or more thereof, wherein the total amount of compound(s) D ranges from 1% to 20% by weight, relative to the total weight of composition (A);
b) applying to the keratin fibers a composition (B) comprising:
i) one or more chemical oxidizing agents chosen from hydrogen peroxide, hydrogen peroxide-generating systems other than peroxygenated salts, or mixtures;
ii) one or more compounds chosen from carbonates, carbonate-generating systems, bicarbonates, bicarbonate-generating systems, or mixtures of two or more thereof;
iii) one or more silicates; and
c) optionally, applying to the keratin fibers a composition (C) comprising one or more compounds D chosen from organic acids, amino acids, chitosans, vitamins B, vitamin C, vanillin, salts thereof, solvates thereof, or mixtures of two or more thereof, wherein the total amount of compound(s) D ranges from 1% to 25% by weight, relative to the total weight of composition (C);
wherein:
at least one of step a) or step c) is present;
when step a) is present, step a) is carried out before step b);
when step c) is present, step c) is carried out after step b);
the compositions (A) and (C) can be identical or different;
the composition (B) comprises a total amount of persulfates of less than 10% by weight, relative to the total amount of composition (B).

29. The method of claim 28, wherein:
the total amount of compound(s) D ranges from 3% to 20% by weight, relative to the total weight of composition (A); and/or
the total amount of compound(s) D ranges from 2% to 15% by weight, relative to the total weight of composition (C).

30. The method of claim 28,
wherein the at least one organic acid is chosen from compounds of formula (I): 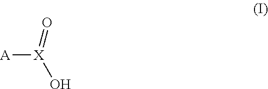
wherein in formula (I):
X represents a carbon atom, S═O, or P—OH;
A represents an —OR⁴ group or a —CR¹(R²)ₚ(R³) group, wherein if X represents P—OH, then A represents an —OR⁴ group;
p=0 or 1;
R¹ represents a hydrogen atom, a hydroxyl (—OH) group, an —NR^{a}—R^{c} group, or an —NR^{a}—CO—R^{c} group;
R² represents a hydrogen atom, a hydroxyl (—OH) group, or an —X(O)OH group with X as defined above;
R³ represents a hydrogen atom, a hydroxyl (—OH) group, or a (hetero)cyclic group optionally substituted by one or more identical or different groups chosen from (C₁-C₆)alkyl; or
R¹ and R³ form, together with the carbon atom which carries them, a (hetero)cyclic group optionally substituted by one or more identical or different groups chosen from: (C₁-C₆)alkyl; or
R¹ and R³ to form, together with the carbon atom which carries them, a carbonyl —CO— group;
R⁴ represents a hydrogen atom, an alkyl group, an alkenyl group, or a (hetero)cyclic group, said alkyl or alkenyl or (hetero)cyclic group being optionally substituted by one or more identical or different groups chosen from: an —X(O)OH group with X as defined above;
R^{a} represents a hydrogen atom or a (C₁-C₄)alkyl group optionally substituted by one or more —X(O)OH groups with X as defined above;
R^{c} represents an alkyl group or an alkenyl group or an alkylamino group or an alkylamido group; and/or
the amino acids are chosen from glycine, proline, methionine, serine, arginine, lysine, carnitine, acetylcarnitine, propionylcarnitine, isovalerylcarnitine, taurine, hypotaurine, creatinine, carnosine, aspartame, or mixtures of two or more thereof; and/or
the vitamins B are chosen from vitamin B1, vitamin B3, vitamin B6, or mixtures thereof.

31. The method of claim 28, wherein the chemical oxidizing agent i) in composition (B) is hydrogen peroxide.

32. The method of claim 28, wherein the total amount of chemical oxidizing agent(s) i) ranges from 1% to 12% by weight, relative to the total weight of the composition (B).

33. The method of claim 28, wherein the total amount of compound(s) ii) ranges from 0.01% to 20% by weight, relative to the total weight of composition (B).

34. The method of claim 28, wherein the one or more compounds ii) is chosen from carbonates, carbonate-generating systems, or mixtures thereof.

35. The method of claim 34, wherein the total amount of carbonate(s) and/or carbonate-generating system(s) ranges from 0.01% to 20% by weight, relative to the total weight of composition (B).

36. The method of claim 28, wherein the carbonate is chosen from:
alkali metal carbonates;
alkaline earth metal carbonates;
lanthanide carbonates;
transition metal carbonates;
bismuth carbonate;
cadmium carbonate;
thallium carbonate;
zinc carbonate;
compounds of formula (N⁺R¹R²R³R⁴)₂CO₃²⁻ wherein R¹, R², R³ and R⁴ are independently chosen from a hydrogen atom or a (C₁-C₄)alkyl group optionally substituted by a hydroxyl group;
guanidine carbonate; or
mixtures of two or more thereof.

37. The method of claim 28, wherein the one or more compounds ii) is chosen from bicarbonates, bicarbonate-generating systems, or mixtures thereof.

38. The method of claim 37, wherein the total amount of bicarbonate(s) and the bicarbonate-generating system(s) ranges from 0.01% to 20% by weight, relative to the total weight of composition (B).

39. The method of claim 28, wherein the bicarbonate is chosen from:
alkali metal bicarbonates;
alkaline earth metal bicarbonates;
compounds of formula N⁺R¹R²R³R⁴HCO₃⁻ wherein R¹, R², R³ and R⁴ are independently chosen from a hydrogen atom or a (C₁-C₄)alkyl group optionally substituted by a hydroxyl group;
aminoguanidine bicarbonate; or
mixtures of two or more thereof.

40. The method of claim 28, wherein the one or more silicates iii) is chosen from alkali metal silicates, alkaline earth metal silicates, aluminium silicates, trimethylammonium silicates, or mixtures of two or more thereof.

41. The method of claim 28, wherein the total amount of silicate(s) iii) ranges from 1% to 40% by weight, relative to the total weight of composition (B).

42. The method of claim 28, wherein the weight ratio of the total amount of bicarbonate(s) and/or bicarbonate-generating system(s) to the total amount of silicate(s) iii) in composition (B) ranges from 1:50 to 7.5:1.

43. The method of claim 28, wherein the weight ratio of the total amount of bicarbonate(s) and/or bicarbonate-generating system(s) to the total amount of chemical oxidizing agents (i) in composition (B) ranges from 11:100 to 5:1.

44. The method of claim 28, wherein the weight ratio of the total amount of carbonate(s) and/or carbonate-generating system(s) to the total amount of bicarbonate(s) and/or bicarbonate-generating system(s) in composition (B) ranges from 3:50 to 20:1.

45. The method of claim 28, wherein composition (B) is made by mixing a composition (B1) with a composition (B2), and optionally a composition (B3), wherein composition (B1) comprises:
one or more chemical oxidizing agents chosen from hydrogen peroxide, hydrogen peroxide-generating systems other than peroxygenated salts, or mixtures thereof; and
wherein composition (B2) comprises:
one or more compounds chosen from carbonates, carbonate-generating systems, bicarbonates, bicarbonate-generating systems, or mixtures thereof; and
one or more silicates; and
optionally one or more coloring agents; and
wherein composition (B3) comprises:
one or more coloring agents.

46. A method for reducing damage to keratin fibers comprising: applying to the keratin fibers a composition comprising:
i) one or more chemical oxidizing agents chosen from hydrogen peroxide, hydrogen peroxide-generating systems other than peroxygenated salts, or mixtures thereof;
ii) one or more compounds chosen from carbonates, carbonate-generating systems, bicarbonates, bicarbonate-generating systems, or mixtures of two or more thereof;
iii) one or more silicates; or
mixtures of two or more thereof.

47. A multicompartment device comprising:
a first compartment comprising a composition (B1) comprising:
one or more chemical oxidizing agents chosen from hydrogen peroxide, hydrogen peroxide-generating systems other than peroxygenated salts, or mixtures thereof; and
a second compartment comprising a composition (B2) comprising:
one or more compounds chosen from carbonates, carbonate-generating systems, bicarbonates, bicarbonate-generating systems, or mixtures thereof; and
one or more silicates;
optionally one or more coloring agents; and
optionally one or more compounds D chosen from organic acids, amino acids, chitosans, vitamins B, vitamin C, vanillin, salts thereof, solvates thereof, or mixtures of two or more thereof; and
a third compartment comprising one of a composition (A) or a composition (C), wherein composition (A) comprises:
one or more compounds D chosen from organic acids, amino acids, chitosans, vitamins B, vitamin C, vanillin, salts thereof, solvates thereof, or mixtures of two or more thereof, wherein the total amount of compounds D ranges from 1% to 20% by weight, relative to the total weight of composition (A); and
optionally one or more coloring agents;
wherein composition (C) comprises:
one or more compounds D chosen from organic acids, amino acids, chitosans, vitamins B, vitamin C, vanillin, salts thereof, solvates thereof, or mixtures of two or more thereof, wherein the total amount of compounds D ranges from 1% to 25% by weight, relative to the total weight of composition (C); and
optionally one or more coloring agents; and
optionally a fourth compartment comprising a composition (B3) comprising one or more coloring agents.
